(19) United States
(12) Patent Application Publication
(10) Pub. No.: US 2022/0313708 A1
(43) Pub. Date: Oct. 6, 2022

(54) COMBINATION PRODUCT CONTAINING LIMONOID COMPOUND AND SGLT-2 INHIBITOR

(71) Applicant: ZHEJIANG YANGSHENGTANG INSTITUTE OF NATURAL MEDICATION CO., LTD., HANGZHOU, ZHEJIANG (CN)

(72) Inventors: Dong LI, HANGZHOU, ZHEJIANG (CN); Quan HAN, HANGZHOU, ZHEJIANG (CN); Liu HU, HANGZHOU, ZHEJIANG (CN); Lian XUE, HANGZHOU, ZHEJIANG (CN)

(21) Appl. No.: 17/633,712

(22) PCT Filed: Jul. 30, 2020
(86) PCT No.: PCT/CN2020/105648

§ 371 Date: Feb. 8, 2022

(30) Foreign Application Priority Data
Aug. 12, 2019 (CN) 201910738827.5

(51) Int. Cl. A61K 31/585; A61K 38/00; A61P 3/10

(52) U.S. Cl. CPC A61K 31/585; A61K 38/005; A61P 3/10

(57) ABSTRACT

The present invention relates to a combination product comprising a limonoid compound (or a pharmaceutically acceptable derivative, ester, stereoisomer, salt or prodrug thereof), and a glucose cotransporter-2 inhibitor (SGLT-2) inhibitor (e.g., canagliflozin, dapagliflozin, empagliflozin, ipragliflozin, luseogliflozin and tofogliflozin). The present invention further relates to a use of the combination product for prevention and/or treatment of a disease associated with diabetes and the like.

### TECHNICAL FIELD

The present invention belongs to the technical field of medicine, and specifically relates to a combination product comprising a limonoid compound (and a pharmaceutically acceptable derivative, ester, stereoisomer, salt or prodrug thereof) and a SGLT-2 inhibitor (or a pharmaceutically acceptable derivative thereof). The present invention also relates to a use of the combination product in the treatment and/or prevention of a disease associated with diabetes and metabolic syndrome.

### BACKGROUND ART

According to IDF statistics, there were about 425 million people with diabetes worldwide in 2017, i.e., 1 out of every 11 people has diabetes. The number of diabetic patients in China is about 110 million, ranking first in the world. It is predicted that by 2040, 642 million people worldwide will have diabetes, and the diabetic patients in China will reach 151 million. Diabetes requires life-long monitoring and treatment, and if not being well controlled, it will lead to secondary cardiovascular diseases, blindness, stroke, diabetic nephropathy, diabetic gangrene and other complications in patients, which will seriously endanger human health and life.

More than 90% of diabetes is type II diabetes, and oral hypoglycemic agents are the main treatment method. At present, the main oral hypoglycemic drugs include: sulfonylureas, biguanides, a-glucosidase inhibitors, thiazolidinediones, SGLT-2 inhibitors, etc., but the oral hypoglycemic drugs are prone to severe side effects such as drug resistance, low blood glucose, and toxicity to liver and kidney.

Regarding SGLT-2 inhibitor, i.e., glucose cotransporter-2 inhibitor, SGLT-2 is mainly expressed in kidney, and about 90% of glucose is reabsorbed through the action of SGLT-2 in proximal convoluted tubule Si segment. In other words, SGLT-2 plays a major role in the reabsorption of glucose, and SGLT-2 transports 90% of the glucose reabsorbed by kidney. Therefore, SGLT-2 inhibitor can block the reabsorption of glucose by the proximal convoluted tubule, while the excess glucose is excreted through urine, thereby achieving the purpose of lowering blood glucose.

The main side effects of SGLT-2 inhibitor are: easy to cause hypoglycemia, ketoacidosis at high doses (including nausea, vomiting, abdominal pain, fatigue and difficulty breathing); urinary tract infection (symptoms such as burning pain when urinating, frequent urination, urinary urgency, etc.; pain under stomach or under pelvis, fever, or hematuria).

The limonoid compounds are mainly present in fruits of rutaceous plants, such as immature bitter orange, navel orange, citrus reticulate, fragrant citrus, pomelo and the like. Their contents are higher in the cores (seeds), and lower in the peel (about 1/10,000 to 5/100,000). About 50 kinds of limonoid compounds have been isolated and identified from citrus plants. The limonoid compounds have various biological activities such as antitumor, insect antifeedant, antiviral, analgesic, anti-inflammatory and hypnotic, and can be used in functional food additives, anti-cancer foods, pesticides, feed additives, etc.

Considering the hypoglycemic effect and side effects of SGLT-2 inhibitors, it is urgent to find a pharmaceutical composition product that is simple to take, good in effect, and low in side effects.

### CONTENTS OF THE INVENTION

The present invention provides a combination product comprising a limonoid compound (or a pharmaceutically acceptable derivative, ester, stereoisomer, salt or prodrug thereof) and a SGLT-2 inhibitor (or a pharmaceutically acceptable derivative thereof), and a use of this composition for preventing or/and treating a disease associated with diabetes and metabolic syndrome. Compared with SGLT-2 inhibitor (or a pharmaceutically acceptable derivative thereof) or limonoid compound (or a pharmaceutically acceptable derivative, ester, stereoisomer, salt or prodrug thereof) as monotherapy at the same dose, the combination product containing a limonoid compound (or a pharmaceutically acceptable derivative, ester, stereoisomer, salt or prodrug thereof) and a SGLT-2 inhibitor (or a pharmaceutically acceptable derivative thereof) as mentioned in the present invention can significantly enhance therapeutic effects such as hypoglycemic effect, and show synergistic effect. At the same time, the amount of SGLT-2 inhibitor (or a pharmaceutically acceptable derivative thereof) is reduced, thereby reducing its side effects.

In a first aspect of the present invention, there is provided a combination product comprising a limonoid compound (or a pharmaceutically acceptable derivative, ester, stereoisomer, salt or prodrug thereof), and a SGLT-2 inhibitor (or a pharmaceutically acceptable derivative), or a combination product comprising only a limonoid compound (or a pharmaceutically acceptable derivative, ester, stereoisomer, salt or prodrug thereof), and a SGLT-2 inhibitor (or a pharmaceutically acceptable derivative) as active ingredients.

The limonoid compound as mentioned in the present invention is a general term for a class of highly oxidized compounds with a 4,4,8-trimethyl-17-furanosteroid skeleton or derivatives thereof (or can be expressed as compounds consisting of variants of furanolactone polycyclic core structure, and having four fused 6-membered rings and one furan ring). Specifically, the examples of the limonoid compound include, but are not limited to: limonin, isolimonic acid, 7α-limonol, obacunone, ichangin, ichangensin, nomilin, deacetylnomilin, nomilin acid, deacetylnomilin acid, citrusin, isoobacunoic acid, etc., and any glycoside derivatives thereof. The structural formula of an exemplary limonoid compound, i.e., limonin, is shown below.

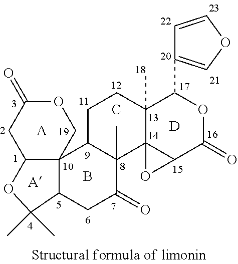

Further, the glucoside derivatives of the limonoid compound as mentioned in the present invention include, but are not limited to: limonin 17-β-glucopyranoiside, ichangin 17-β-D-glucopyranoiside, isolimonic acid 17-β-D-glucopyranoside, deactylnomilin 17-β-Dglucopyranoside, nomilin 17-β-D-glucopyranoside, obacunone 17-β-D-glucopynoside, nomilinic acid 17-β-D-glucopyranosid, deacetylnomilinic acid 17-β-D-glucopyranosid, etc.

In some embodiments, the limonoid compound as mentioned in the present invention is in the form of a monomer or an extract. The monomer is extracted or artificially synthesized, and its sources may be commercially available, or they can be easily prepared and obtained by the prior art in the art.

The SGLT-2 inhibitor mentioned in the present invention includes but is not limited to canagliflozin, dapagliflozin, empagliflozin, ipragliflozin, luseogliflozin and tofogliflozin. Further, the SGLT-2 inhibitor is canagliflozin, empagliflozin, ipragliflozin and tofogliflozin. The SGLT-2 inhibitor can exist in the form of original compound or in the form of a pharmaceutically acceptable derivative thereof.

In some embodiments, the combination product is in the form of a pharmaceutical composition, and the pharmaceutical composition is in a unit dosage form.

In some embodiments, the limonoid compound (or a pharmaceutically acceptable derivative, ester, stereoisomer, salt, or prodrug thereof) and the SGLT-2 inhibitor (or a pharmaceutically acceptable derivative thereof) are each in the form of a separate preparation. Further, the limonoid compound (or a pharmaceutically acceptable derivative, ester, stereoisomer, salt or prodrug thereof) and the SGLT-2 inhibitor (or a pharmaceutically acceptable derivative thereof) are each in the form of a separate unit dose. Further, the limonoid compound (or a pharmaceutically acceptable derivative, ester, stereoisomer, salt or prodrug thereof) and the SGLT-2 inhibitor (or a pharmaceutically acceptable derivative thereof) can be administered simultaneously or sequentially.

In some embodiments, the SGLT-2 inhibitor (or a pharmaceutically acceptable derivative thereof) has an amount of 10 mg, 20 mg, 40 mg, 50 mg, 75 mg, 100 mg, 150 mg, 200 mg, 250 mg, 300 mg, 375 mg, 500 mg, 750 mg, 1500 mg, 1875 mg, or 2000 mg, and the ranges between these amounts, wherein the ranges include but are not limited to: 10 mg to 20 mg, 10 mg to 40 mg, 10 mg to 50 mg, 10 mg to 75 mg, 10 mg to 100 mg, 10 mg to 150 mg, 10 mg to 200 mg, 10 mg to 250 mg, 10 mg to 300 mg, 10 mg to 375 mg, 10 mg to 500 mg, 10 mg to 750 mg, 10 mg to 1500 mg, 10 mg to 1875 mg, 10 mg to 2000 mg, 20 mg to 40 mg, 20 mg to 50 mg, 20 mg to 75 mg, 20 mg to 100 mg, 20 mg to 150 mg, 20 mg to 200 mg, 20 mg to 250 mg, 20 mg to 300 mg, 20 mg to 375 mg, 20 mg to 500 mg, 20 mg to 750 mg, 20 mg to 1500 mg, 20 mg to 1875 mg, 20 mg to 2000 mg, 40 mg to 50 mg, 40 mg to 75 mg, 40 mg to 100 mg, 40 mg to 150 mg, 40 mg to 200 mg, 40 mg to 250 mg, 40 mg to 300 mg, 40 mg to 375 mg, 40 mg to 500 mg, 40 mg to 750 mg, 40 mg to 1500 mg, 40 mg to 1875 mg, 40 mg to 2000 mg, 50 mg to 75 mg, 50 mg to 100 mg, 50 mg to 150 mg, 50 mg to 200 mg, 50 mg to 250 mg, 50 mg to 300 mg, 50 mg to 375 mg, 50 mg to 500 mg, 50 mg to 750 mg, 50 mg to 1500 mg, 50 mg to 1875 mg, 50 mg to 2000 mg, 75 mg to 100 mg, 75 mg to 150 mg, 75 mg to 200 mg, 75 mg to 250 mg, 75 mg to 300 mg, 75 mg to 375 mg, 75 mg to 500 mg, 75 mg to 750 mg, 75 mg to 1500 mg, 75 mg to 1875 mg, 75 mg to 2000 mg, 100 mg to 150 mg, 100 mg to 200 mg, 100 mg to 250 mg, 100 mg to 300 mg, 100 mg to 375 mg, 100 mg to 500 mg, 100 mg to 750 mg, 100 mg to 1500 mg, 100 mg to 1875 mg, 100 mg to 2000 mg, 150 mg to 200 mg, 150 mg to 250 mg, 150 mg to 300 mg, 150 mg to 375 mg, 150 mg to 500 mg, 150 mg to 750 mg, 150 mg to 1500 mg, 150 mg to 1875 mg, 150 mg to 2000 mg, 200 mg to 250 mg, 200 mg to 300 mg, 200 mg to 375 mg, 200 mg to 500 mg, 200 mg to 750 mg, 200 mg to 1500 mg, 200 mg to 1875 mg, 200 mg to 2000 mg, 250 mg to 300 mg, 250 mg to 375 mg, 250 mg to 500 mg, 250 mg to 750 mg, 250 mg to 1500 mg, 250 mg to 1875 mg, 250 mg to 2000 mg, 300 mg to 375 mg, 300 mg to 500 mg, 300 mg to 750 mg, 300 mg to 1500 mg, 300 mg to 1875 mg, 300 mg to 2000 mg, 375 mg to 500 mg, 375 mg to 750 mg, 375 mg to 1500 mg, 375 mg to 1875 mg, 375 mg to 2000 mg, 500 mg to 750 mg, 500 mg to 1500 mg, 500 mg to 1875 mg, 500 mg to 2000 mg, 750 mg to 1500 mg, 750 mg to 1875 mg, 750 mg to 2000 mg, and 1875 mg to 2000 mg.

In some embodiments, the limonoid compound (or a pharmaceutically acceptable derivative, ester, stereoisomer, salt or prodrug thereof) has an amount of 10 mg, 20 mg, 40 mg, 50 mg, 75 mg, 100 mg, 150 mg, 200 mg, 250 mg, 300 mg, 375 mg, 500 mg, 750 mg, 1500 mg, 1875 mg, or 2000 mg, and the ranges between these amounts, wherein the ranges include but are not limited to: 10 mg to 20 mg, 10 mg to 40 mg, 10 mg to 50 mg, 10 mg to 75 mg, 10 mg to 100 mg, 10 mg to 150 mg, 10 mg to 200 mg, 10 mg to 250 mg, 10 mg to 300 mg, 10 mg to 375 mg, 10 mg to 500 mg, 10 mg to 750 mg, 10 mg to 1500 mg, 10 mg to 1875 mg, 10 mg to 2000 mg, 20 mg to 40 mg, 20 mg to 50 mg, 20 mg to 75 mg, 20 mg to 100 mg, 20 mg to 150 mg, 20 mg to 200 mg, 20 mg to 250 mg, 20 mg to 300 mg, 20 mg to 375 mg, 20 mg to 500 mg, 20 mg to 750 mg, 20 mg to 1500 mg, 20 mg to 1875 mg, 20 mg to 2000 mg, 40 mg to 50 mg, 40 mg to 75 mg, 40 mg to 100 mg, 40 mg to 150 mg, 40 mg to 200 mg, 40 mg to 250 mg, 40 mg to 300 mg, 40 mg to 375 mg, 40 mg to 500 mg, 40 mg to 750 mg, 40 mg to 1500 mg, 40 mg to 1875 mg, 40 mg to 2000 mg, 50 mg to 75 mg, 50 mg to 100 mg, 50 mg to 150 mg, 50 mg to 200 mg, 50 mg to 250 mg, 50 mg to 300 mg, 50 mg to 375 mg, 50 mg to 500 mg, 50 mg to 750 mg, 50 mg to 1500 mg, 50 mg to 1875 mg, 50 mg to 2000 mg, 75 mg to 100 mg, 75 mg to 150 mg, 75 mg to 200 mg, 75 mg to 250 mg, 75 mg to 300 mg, 75 mg to 375 mg, 75 mg to 500 mg, 75 mg to 750 mg, 75 mg to 1500 mg, 75 mg to 1875 mg, 75 mg to 2000 mg, 100 mg to 150 mg, 100 mg to 200 mg, 100 mg to 250 mg, 100 mg to 300 mg, 100 mg to 375 mg, 100 mg to 500 mg, 100 mg to 750 mg, 100 mg to 1500 mg, 100 mg to 1875 mg, 100 mg to 2000 mg, 150 mg to 200 mg, 150 mg to 250 mg, 150 mg to 300 mg, 150 mg to 375 mg, 150 mg to 500 mg, 150 mg to 750 mg, 150 mg to 1500 mg, 150 mg to 1875 mg, 150 mg to 2000 mg, 200 mg to 250 mg, 200 mg to 300 mg, 200 mg to 375 mg, 200 mg to 500 mg, 200 mg to 750 mg, 200 mg to 1500 mg, 200 mg to 1875 mg, 200 mg to 2000 mg, 250 mg to 300 mg, 250 mg to 375 mg, 250 mg to 500 mg, 250 mg to 750 mg, 250 mg to 1500 mg, 250 mg to 1875 mg, 250 mg to 2000 mg, 300 mg to 375 mg, 300 mg to 500 mg, 300 mg to 750 mg, 300 mg to 1500 mg, 300 mg to 1875 mg, 300 mg to 2000 mg, 375 mg to 500 mg, 375 mg to 750 mg, 375 mg to 1500 mg, 375 mg to 1875 mg, 375 mg to 2000 mg, 500 mg to 750 mg, 500 mg to 1500 mg, 500 mg to 1875 mg, 500 mg to 2000 mg, 750 mg to 1500 mg, 750 mg to 1875 mg, 750 mg to 2000 mg, and 1875 mg to 2000 mg.

In some embodiments, the SGLT-2 inhibitor is selected from canagliflozin, dapagliflozin, empagliflozin, ipragliflozin, luseogliflozin and tofogliflozin, and the limonoid compound is one or more selected from: limonin, isolimonic acid, 7α-limonol, obacunone, ichangin, ichangensin, nomilin, deacetylnomilin, nomilin acid, deacetylnomilin acid, citrusin, isoobacunoic acid, etc., and any glycoside derivatives thereof.

In some embodiments, the combination product further comprises a pharmaceutically acceptable carrier, diluent, or excipient.

In some embodiments, the combination product is in the form of tablet, capsule, granule, syrup, powder, lozenge, sachet, cachet, elixir, suspension, emulsion, solution, syrup, aerosol, ointment, cream and injection.

In a second aspect of the present invention, there is provided a use of the combination product in manufacture of a medicament for the prevention and/or treatment of a disease associated with diabetes and metabolic syndrome. In some embodiments, the diabetes is type I diabetes. In some embodiments, the diabetes is type II diabetes.

In a third aspect of the present invention, there is provided a method of administering the limonoid compound (or a pharmaceutically acceptable derivative, ester, stereoisomer, salt or prodrug thereof) and the SGLT-2 inhibitor (or a pharmaceutically acceptable derivative thereof) in combination to prevent and/or treat a disease. In some embodiments, there is provided a method of administering the limonoid compound (or a pharmaceutically acceptable derivative, ester, stereoisomer, salt or prodrug thereof) and the SGLT-2 inhibitor (or a pharmaceutically acceptable derivative thereof) in combination to prevent and/or treat a disease associated with diabetes and metabolic syndrome. In some embodiments, there is provided a method of administering the limonoid compound (or a pharmaceutically acceptable derivative, ester, stereoisomer, salt or prodrug thereof) and the SGLT-2 inhibitor (or a pharmaceutically acceptable derivative thereof) in combination to lower a blood glucose. In some embodiments, there is provided a method of administering the limonoid compound (or a pharmaceutically acceptable derivative, ester, stereoisomer, salt or prodrug thereof) and the SGLT-2 inhibitor (or a pharmaceutically acceptable derivative thereof) in combination to improve an insulin sensitivity. In some embodiments, there is provided a method of administering the limonoid compound (or a pharmaceutically acceptable derivative, ester, stereoisomer, salt or prodrug thereof) and the SGLT-2 inhibitor (or a pharmaceutically acceptable derivative thereof) in combination to improve a leptin sensitivity.

In some embodiments, the limonoid compound (or a pharmaceutically acceptable derivative, ester, stereoisomer, salt or prodrug thereof) and the SGLT-2 inhibitor (or a pharmaceutically acceptable derivative thereof) can be mixed into a preparation and administered in the form of a pharmaceutical composition (preferably, a dosage unit form); in some embodiments, the limonoid compound (or a pharmaceutically acceptable derivative, ester, stereoisomer, salt or prodrug thereof) and the SGLT-2 inhibitor (or a pharmaceutically acceptable derivative thereof) are each in separate preparation form (preferably, each in separate dosage unit form) and separately administered; in some embodiments, the limonoid compound (or a pharmaceutically acceptable derivative, ester, stereoisomer, salt or prodrug thereof) and the SGLT-2 inhibitor (or a pharmaceutically acceptable derivative thereof) are administered simultaneously; in some embodiments, the limonoid compound (or a pharmaceutically acceptable derivative, ester, stereoisomer, salt or prodrug thereof) and the SGLT-2 inhibitor (or a pharmaceutically acceptable derivative thereof) are administered one after another; in some embodiments, the limonoid compound (or a pharmaceutically acceptable derivative, ester, stereoisomer, salt or prodrug thereof) and the SGLT-2 inhibitor (or a pharmaceutically acceptable derivative thereof) are administered one after another at a time interval of about 30 minutes, or about 1 hour, or about 2 hours, or about 4 hours, or about 8 hours, or about 12 hours. In some embodiments, as required, the combination product comprising the limonoid compound (or a pharmaceutically acceptable derivative, ester, stereoisomer, salt or prodrug thereof) and the SGLT-2 inhibitor (or a pharmaceutically acceptable derivative thereof) according to the present invention that is in the form of pharmaceutical composition (preferably, a dosage unit form) is administered for, including, but are not limited to: 1, 2, 3, 4, 5 or 6 times per day. In some embodiments, as required, the combination product comprising the limonoid compound (or a pharmaceutically acceptable derivative, ester, stereoisomer, salt or prodrug thereof) and the SGLT-2 inhibitor (or a pharmaceutically acceptable derivative thereof) according to the present invention that are each in separate preparation form (preferably, each in separate dosage unit form) is administered for, including, but are not limited to: 1, 2, 3, 4, 5 or 6 times per day.

In some embodiments, the limonin compound (or a pharmaceutically acceptable derivative, ester, stereoisomer, salt or prodrug thereof), and the SGLT-2 inhibitor (or a pharmaceutically acceptable derivative) or the combination product comprising them can be administered by the following administration modes, for example, oral administration, injection administration (e.g., subcutaneous and parenteral administration) and topical administration.

In some embodiments, the limonin compound (or a pharmaceutically acceptable derivative, ester, stereoisomer, salt, or prodrug thereof), and the SGLT-2 inhibitor (or a pharmaceutically acceptable derivative thereof) have a daily dosage as follows: as calculated according to adult body weight of 60 kg, the daily dosage of the SGLT-2 inhibitor (or a pharmaceutically acceptable derivative thereof) is 10 mg, 20 mg, 40 mg, 50 mg, 75 mg, 100 mg, 150 mg, 200 mg, 250 mg, 300 mg, 375 mg, 500 mg, 750 mg, 1500 mg, 1875 mg or 2000 mg, and the ranges between these dosages, wherein the ranges include but are not limited to: 10 mg to 20 mg, 10 mg to 40 mg, 10 mg to 50 mg, 10 mg to 75 mg, 10 mg to 100 mg, 10 mg to 150 mg, 10 mg to 200 mg, 10 mg to 250 mg, 10 mg to 300 mg, 10 mg to 375 mg, 10 mg to 500 mg, 10 mg to 750 mg, 10 mg to 1500 mg, 10 mg to 1875 mg, 10 mg to 2000 mg, 20 mg to 40 mg, 20 mg to 50 mg, 20 mg to 75 mg, 20 mg to 100 mg, 20 mg to 150 mg, 20 mg to 200 mg, 20 mg to 250 mg, 20 mg to 300 mg, 20 mg to 375 mg, 20 mg to 500 mg, 20 mg to 750 mg, 20 mg to 1500 mg, 20 mg to 1875 mg, 20 mg to 2000 mg, 40 mg to 50 mg, 40 mg to 75 mg, 40 mg to 100 mg, 40 mg to 150 mg, 40 mg to 200 mg, 40 mg to 250 mg, 40 mg to 300 mg, 40 mg to 375 mg, 40 mg to 500 mg, 40 mg to 750 mg, 40 mg to 1500 mg, 40 mg to 1875 mg, 40 mg to 2000 mg, 50 mg to 75 mg, 50 mg to 100 mg, 50 mg to 150 mg, 50 mg to 200 mg, 50 mg to 250 mg, 50 mg to 300 mg, 50 mg to 375 mg, 50 mg to 500 mg, 50 mg to 750 mg, 50 mg to 1500 mg, 50 mg to 1875 mg, 50 mg to 2000 mg, 75 mg to 100 mg, 75 mg to 150 mg, 75 mg to 200 mg, 75 mg to 250 mg, 75 mg to 300 mg, 75 mg to 375 mg, 75 mg to 500 mg, 75 mg to 750 mg, 75 mg to 1500 mg, 75 mg to 1875 mg, 75 mg to 2000 mg, 100 mg to 150 mg, 100 mg to 200 mg, 100 mg to 250 mg, 100 mg to 300 mg, 100 mg to 375 mg, 100 mg to 500 mg, 100 mg to 750 mg, 100 mg to 1500 mg, 100 mg to 1875 mg, 100 mg to 2000 mg, 150 mg to 200 mg, 150 mg to 250 mg, 150 mg to 300 mg, 150 mg to 375 mg, 150 mg to 500 mg, 150 mg to 750 mg, 150 mg to 1500 mg, 150 mg to 1875 mg, 150 mg to 2000 mg, 200 mg to 250 mg, 200 mg to 300 mg, 200 mg to 375 mg, 200 mg to 500 mg, 200 mg to 750 mg, 200 mg to 1500 mg, 200 mg to 1875 mg, 200 mg to 2000 mg, 250 mg to 300 mg, 250 mg to 375 mg, 250 mg to 500 mg, 250 mg to 750 mg, 250 mg to 1500 mg, 250 mg to 1875 mg, 250 mg to 2000 mg, 300 mg to 375 mg, 300 mg to 500 mg, 300 mg to 750 mg, 300 mg to 1500 mg, 300 mg to 1875 mg, 300 mg to 2000 mg, 375 mg to 500 mg, 375 mg to 750 mg, 375 mg to 1500 mg, 375 mg to 1875 mg, 375 mg to 2000 mg, 500 mg to 750 mg, 500 mg to 1500 mg, 500 mg to 1875 mg, 500 mg to 2000 mg, 750 mg to 1500 mg, 750 mg to 1875 mg, 750 mg to 2000 mg and 1875 mg to 2000 mg. As calculated according to adult body weight of 60 kg, the daily dosage of the limonoid compound (or a pharmaceutically acceptable derivative, ester, stereoisomer, salt or prodrug thereof) is 10 mg, 20 mg, 40 mg, 50 mg, 75 mg, 100 mg, 150 mg, 200 mg, 250 mg, 300 mg, 375 mg, 500 mg, 750 mg, 1500 mg, 1875 mg or 2000 mg, and the ranges of between these dosages, wherein the ranges includes but are not limited to: 10 mg to 20 mg, 10 mg to 40 mg, 10 mg to 50 mg, 10 mg to 75 mg, 10 mg to 100 mg, 10 mg to 150 mg, 10 mg to 200 mg, 10 mg to 250 mg, 10 mg to 300 mg, 10 mg to 375 mg, 10 mg to 500 mg, 10 mg to 750 mg, 10 mg to 1500 mg, 10 mg to 1875 mg, 10 mg to 2000 mg, 20 mg to 40 mg, 20 mg to 50 mg, 20 mg to 75 mg, 20 mg to 100 mg, 20 mg to 150 mg, 20 mg to 200 mg, 20 mg to 250 mg, 20 mg to 300 mg, 20 mg to 375 mg, 20 mg to 500 mg, 20 mg to 750 mg, 20 mg to 1500 mg, 20 mg to 1875 mg, 20 mg to 2000 mg, 40 mg to 50 mg, 40 mg to 75 mg, 40 mg to 100 mg, 40 mg to 150 mg, 40 mg to 200 mg, 40 mg to 250 mg, 40 mg to 300 mg, 40 mg to 375 mg, 40 mg to 500 mg, 40 mg to 750 mg, 40 mg to 1500 mg, 40 mg to 1875 mg, 40 mg to 2000 mg, 50 mg to 75 mg, 50 mg to 100 mg, 50 mg to 150 mg, 50 mg to 200 mg, 50 mg to 250 mg, 50 mg to 300 mg, 50 mg to 375 mg, 50 mg to 500 mg, 50 mg to 750 mg, 50 mg to 1500 mg, 50 mg to 1875 mg, 50 mg to 2000 mg, 75 mg to 100 mg, 75 mg to 150 mg, 75 mg to 200 mg, 75 mg to 250 mg, 75 mg to 300 mg, 75 mg to 375 mg, 75 mg to 500 mg, 75 mg to 750 mg, 75 mg to 1500 mg, 75 mg to 1875 mg, 75 mg to 2000 mg, 100 mg to 150 mg, 100 mg to 200 mg, 100 mg to 250 mg, 100 mg to 300 mg, 100 mg to 375 mg, 100 mg to 500 mg, 100 mg to 750 mg, 100 mg to 1500 mg, 100 mg to 1875 mg, 100 mg to 2000 mg, 150 mg to 200 mg, 150 mg to 250 mg, 150 mg to 300 mg, 150 mg to 375 mg, 150 mg to 500 mg, 150 mg to 750 mg, 150 mg to 1500 mg, 150 mg to 1875 mg, 150 mg to 2000 mg, 200 mg to 250 mg, 200 mg to 300 mg, 200 mg to 375 mg, 200 mg to 500 mg, 200 mg to 750 mg, 200 mg to 1500 mg, 200 mg to 1875 mg, 200 mg to 2000 mg, 250 mg to 300 mg, 250 mg to 375 mg, 250 mg to 500 mg, 250 mg to 750 mg, 250 mg to 1500 mg, 250 mg to 1875 mg, 250 mg to 2000 mg, 300 mg to 375 mg, 300 mg to 500 mg, 300 mg to 750 mg, 300 mg to 1500 mg, 300 mg to 1875 mg, 300 mg to 2000 mg, 375 mg to 500 mg, 375 mg to 750 mg, 375 mg to 1500 mg, 375 mg to 1875 mg, 375 mg to 2000 mg, 500 mg to 750 mg, 500 mg to 1500 mg, 500 mg to 1875 mg, 500 mg to 2000 mg, 750 mg to 1500 mg, 750 mg to 1875 mg, 750 mg to 2000 mg and 1875 mg to 2000 mg.

In a fourth aspect of the present invention, there is provided a method for preparing a combination product in the form of a pharmaceutical composition. In order to improve its operability as a drug or its absorbability when used in a living body, the limonoid compound or a pharmaceutically acceptable derivative, ester, stereoisomer, salt or prodrug thereof and the SGLT-2 inhibitor or a pharmaceutically acceptable derivative thereof are preferably combined with a pharmaceutical adjuvant such as a pharmaceutically acceptable carrier, excipient, diluent, etc., so as to form a preparation, thereby obtaining the form.

In a fifth aspect of the invention, there is provided a kit, the kit comprising the combination product described herein.

The term “pharmaceutically acceptable salt” as used throughout this description refers to a salt of a free acid or a free base, that is typically prepared by reacting the free base with a suitable organic or inorganic acid or by reacting the acid with a suitable organic or inorganic base. The term can be used for any compound, including limonoid compounds (having the function of free acid or free base) and the like. Representative salts include: acetate, benzenesulfonate, benzoate, bicarbonate, bisulfate, hydrogen tartrate, borate, bromide, calcium edetate, camphorsulfonate, carbonate, chloride, clavulanate, citrate, dihydrochloride, edetate, ethanedisulfonate, estolate, esylate, fumarate, glucoheptonate, gluconate, glutamate, glycol lylarsanilate, hexylresorcinate, hydrabamine, hydrobromide, hydrochloride, hydroxynaphthoate, iodide, isethionate, lactate, lactobionate, laurate, malate, maleate, mandelate, methanesulfonate, methobromate, methonitrate, methosulfate, monopotassium maleate, mucate, naphthalenesulfonate, nitrate, N-methylglucosamine salt, oxalate, pamoate, palmitate, pantothenate, phosphate/bisphosphate, polygalacturonate, potassium salt, salicylate, sodium salt, stearate, subacetate, succinate, tannate, tartrate, teoclate, p-toluenesulfonate, triethiodide, trimethylamine salt, and valerate. When an acidic substituent is present, for example, —COOH, an ammonium salt, morpholine salt, sodium salt, potassium salt, barium salt, calcium salt, and the like can be formed for use in a dosage form. When a basic group, for example an amino group or a basic heteroaryl group such as pyridyl, is present, an acidic salt such as a hydrochloride, hydrobromide, phosphate, sulfate, trifluoroacetate, trichloroacetate, acetate, oxalate, maleate, pyruvate, malonate, succinate, citrate, tartrate, fumarate, mandelate, benzoate, cinnamate, mesylate, ethanesulfonate, picrate, etc.

The sources of the SGLT-2 inhibitor referred to in the present invention may include, but are not limited to: canagliflozin single tablets, dapagliflozin single tablets, empagliflozin single tablets, proline-ipragliflozin single tablets, luseogliflozin single tablets, tofogliflozin single tablets, canagliflozin/metformin tablets, dapagliflozin/metformin tablets, empagliflozin/metformin tablets, proline-ipragliflozin/metformin tablets, luseogliflozin/metformin tablets, tofogliflozin/metformin tablets, dapagliflozin/dapagliflozin tablets, empagliflozin/ipragliflozin tablets, empagliflozin/ipragliflozin tablets and so on.

### SPECIFIC MODELS FOR CARRYING OUT THE INVENTION

The present invention is further described below through specific examples and comparative examples. However, it should be understood that these examples and comparative examples are only used for more detailed and specific explanation, and should not be understood as limiting the present invention in any form.

In the examples of the present invention, the following diabetic mouse models (the models were well known to those skilled in the art or were easily available according to conventional textbooks, technical manuals, and scientific literature in the art) were used to simulate the pathological conditions of different stages of diabetes in humans. The limonoid compound mentioned in the examples was present in the form of a monomer or an extract. The monomer was extracted or artificially synthesized, and its sources were commercially available, or it could be easily prepared and obtained by the prior art in the art.

### EXAMPLE 1

Effects of limonoid compound, canagliflozin or combination thereof on blood glucose in mouse pancreatic islet β-cell injury model.

In this example, a mouse pancreatic islet β-cell injury model was established by modeling ICR mice with streptozotocin (STZ) (Li Nan et al., Protective effect of pine pollen on kidney damage in diabetic nephropathy mice, Science and Technology Review, 2014, 32 (4/5): 95-99), and used to complete the evaluation of hypoglycemic effect in animals (this model could simulate pancreatic islet β-cell damage state of type I and type II diabetics). The limonoid compound was selected from the group consisting of limonin, isolimonic acid, limonin glycoside, and isolimonic acid glycoside, and canagliflozin single administration group, limonin single administration group, isolimonic acid single administration group, limonin glycoside singe administration group, isolimonic acid glycoside singe administration group, and respective combination thereof with canagliflozin administration groups were set.

Conditions of experimental feeding: ICR mice (20±2 g), aged 6 weeks, purchased from Zhejiang Academy of Medical Sciences, and subjected to experimental feeding after 7 days of preliminary feeding. It should be noted that the conditions for raising the mice were as follows: the temperature was 23±1° C., the humidity was 55±10%, the lights were turned on between 7 am and 7 pm (the lights were turned off at other time), and the mice were allowed to freely take in water and feed. The experimental feed was mouse growth-stable feed (GB M2118), and the daily feeding and management of the animals were under the responsibility of the animal security department, which provided the animals with sufficient diet and fresh drinking water daily.

Experimental grouping: 15 male mice were randomly selected as the normal control group. After fasting for 12 hours, the remaining mice were intraperitoneally injected once with STZ at a dose of 150 mg/kg, and 72 hours later, the mice with blood glucose value of 15 to 25 mmol/L were undifferentiatedly grouped and used in the experiment, 15 animals in each group, and subjected to blood sampling and detection of indicators after two weeks of administration.

Gavage doses: the gavage dose was 0.02 g/kg per day for the limonin group, the gavage dose was 0.02 g/kg per day for the isolimonic acid group, the gavage dose was 0.02 g/kg per day for the limonin glycoside group, the gavage dose was 0.02 g/kg per day for the isolimonic acid glycoside group, the gavage dose of canagliflozin was 0.02 g/kg per day for the canagliflozin group, limonin at a dose of 0.01 g/kg and canagliflozin at a dose of 0.01 g/kg were simultaneously gavaged per day for the limonin/canagliflozin combination group, isolimonic acid at a dose of 0.01 g/kg and canagliflozin at a dose of 0.01 g/kg were simultaneously gavaged per day for the isolimonic acid/canagliflozin combination group, limonin glycoside at a dose of 0.01 g/kg and canagliflozin at a dose of 0.01 g/kg were simultaneously gavaged per day for the limonin glycoside/canagliflozin combination group, isolimonic acid glycoside at a dose of 0.01 g/kg and canagliflozin at a dose of 0.01 g/kg were simultaneously gavaged per day for the isolimonic acid glycoside/canagliflozin combination group, the gavage volume was 10 mL/kg, and the normal group and the model group were administrated with 10 mL/kg of distilled water. Two weeks later, the blood glucose values were measured by tail trimming method (Johnson's blood glucose meter) 1 h after the last administration, and the average of each group was obtained. SPSS 16.0 software was used for statistical analysis. The data were expressed as mean and standard deviation. The data before and after were analyzed by t-test, and P<0.05 was considered statistically significant. The test results were shown in Table 1 below.

TABLE 1
Blood glucose values of STZ mice after two weeks of daily intragastric gavage.

| | Formulation and dosage of | Blood glucose |
|---|---|---|
| Group | administration | value (mmol/L) |
| Normal control group | None | 7.5 ± 0.6 |
| Model group | None | 29.5 ± 4.6 |
| Canagliflozin group | Canagliflozin 0.02 g/kg | 19.1 ± 4.1** |
| Limonin group | Limonin 0.02 g/kg | 19.5 ± 1.8** |
| Isolimonic acid group | Isolimonic acid 0.02 g/kg | 18.2 ± 3.1** |
| Limonin glycoside group | Limonin glycoside 0.02 g/kg | 18.9 ± 2.2** |
| Isolimonic acid glycoside group | Isolimonic acid glycoside 0.02 g/kg | 17.9 ± 2.8** |
| Limonin combination group | Limonin 0.01 g/kg + | 8.1 ± 2.3** |
| | Canagliflozin 0.01 g/kg | |
| Isolimonic acid combination | Isolimonic acid 0.01 g/kg + | 8.4 ± 2.0** |
| group | Canagliflozin 0.01 g/kg | |
| Limonin glycoside combination | Limonin glycoside 0.01 g/kg + | 8.3 ± 1.5** |
| | Canagliflozin 0.01 g/kg | |
| Isolimonic acid glycoside | Isolimonic acid glycoside 0.01 g/kg + | 8.2 ± 1.9** |
| combination group | Canagliflozin 0.01 g/kg | |

Note
*After independent t-test, compared with the model group, the difference was extremely significant (P < 0.05)
**After independent t-test, compared with the model group, the difference was extremely significant (P < 0.01)

Discussion of Experimental Results

From the above results, it could be seen that, compared with the model group, either in single administration or in combination administration with canagliflozin, limonin and its derivatives could significantly reduce the blood glucose values of the mice with STZ islet cell injury. The administration of limonin and its derivatives in combination with canagliflozin had significantly improved the effect as compared with their single administration, showing a synergistic effect. In addition, when limonin and its derivatives were administrated in combination with canagliflozin, as compared with their single administration, the doses of both could be effectively reduced while comparable glucose-lowering effects could still be achieved, which improved the safety of therapeutic regimen and reduced side effects.

### EXAMPLE 2

Effects of limonoid compound, dapagliflozin or combination thereof on blood glucose and leptin in mouse type II diabetes model.

In the present example, db/db mice (line name BKS.Cg-Dock7^{m+/+}Lepr^{db}/Nju) were used to perform hypoglycemic efficacy evaluation test of animals (blood glucose level and leptin). The limonoid compound was selected from obacunone, isoobacunoic acid and obacunone glycoside, and dapagliflozin single administration group, obacunone single administration group, isoobacunoic acid single administration group, obacunone glycoside single administered group, and respective combination thereof with dapagliflozin administration groups were set.

Conditions for experimental feeding: as type II diabetes model mice, 6-week-old SPF-grade db/db mice were purchased from the Nanjing Model Biology Institute, and subjected to experimental feeding after 7 days of preliminary feeding. It should be noted that the conditions for raising the mice were as follows: the temperature was 23±1° C., the humidity was 55±10%, the lights were turned on between 7 am and 7 pm (the lights were turned off at other time), and the mice were allowed to freely take in water and feed. The experimental feed was mouse growth-stable feed (GB M2118), and the daily feeding and management of the animals were under the responsibility of the animal security department, which provided the animals with sufficient diet and fresh drinking water daily.

Experimental grouping: male db/db mice (20±2 g) were selected, and 18 male mice in each group were tested. The experimental groups included normal control group (db/m, n=18), model group (db/db, n=18), obacunone group (db/db, n=18), isoobacunoic acid group (db/db, n=18), obacunone glycoside group (db/db, n=18), dapagliflozin group (db/db, n=18), obacunone/dapagliflozin combination group (db/db, n=18), isoobacunoic acid/dapagliflozin combination group (db/db, n=18), obacunone glycoside/dapagliflozin combination group (db/db, n=18).

Gavage doses: obacunone at a dose of 0.04 g/kg was gavaged per day for the obacunone group, isoobacunoic acid at a dose of 0.04 g/kg was gavaged per day for the isoobacunoic acid group, obacunone glycoside at a dose of 0.04 g/kg was gavaged per day for the obacunone glycoside group, dapagliflozin at a dose of 0.04 g/kg was gavaged per day for the dapagliflozin group, obacunone at a dose of 0.02 g/kg and dapagliflozin at a dose of 0.02 g/kg were simultaneously gavaged per day for the obacunone/dapagliflozin combination group, isoobacunoic acid at a dose of 0.02 g/kg and dapagliflozin at a dose of 0.02 g/kg were simultaneously gavaged per day for the isoobacunoic acid/dapagliflozin combination group, obacunone glycoside at a dose of 0.02 g/kg and dapagliflozin at a dose of 0.02 g/kg were simultaneously gavaged per day for the obacunone glycoside/dapagliflozin combination group, the gavage volume was 10 mL/kg, and the normal group and the model group were administrated with 10 mL/kg of distilled water. Two weeks later, the blood glucose values were measured by tail trimming method (Johnson's blood glucose meter) 1 h after the last administration, and serum leptin levels were measured with blood collected from orbital cavity by enzyme-linked immunosorbent assay (Elisa), and the average of each group was obtained. SPSS 16.0 software was used for statistical analysis. The data were expressed as mean and standard deviation. The data before and after were analyzed by t-test, and P<0.05 was considered statistically significant. The test results were shown in Table 2 below.

TABLE 2
Blood glucose values and leptin of db/db mice after two weeks of daily gavage.

| | Formulation and dosage of | Blood glucose | Leptin |
|---|---|---|---|
| Group | administration | value (mmol/L) | (pg/ml) |
| Normal control group | None | 6.0 ± 0.7 | 0.88 ± 0.18 |
| Model group | None | 23.9 ± 4.2 | 48.19 ± 8.12 |
| Obacunone group | Obacunone 0.04 g/kg | 12.7 ± 2.8** | 25.99 ± 3.92** |
| Isoobacunoic acid group | Isoobacunoic acid 0.04 g/kg | 14.6 ± 3.1** | 27.68 ± 3.25** |
| Obacunone glycoside | Obacunone glycoside 0.04 g/kg | 13.6 ± 2.9** | 28.55 ± 4.00** |
| group | | | |
| Dapagliflozin group | Dapagliflozin 0.04 g/kg | 12.0 ± 2.9** | 25.34 ± 5.26** |
| Obacunone combination | Obacunone 0.02 g/kg + | 6.7 ± 1.0** | 13.21 ± 2.11** |
| group | Dapagliflozin 0.02 g/kg | | |
| Isoobacunoic acid | Isoobacunoic acid 0.02 g/kg + | 6.9 ± 2.1** | 13.97 ± 3.73** |
| combination group | Dapagliflozin 0.02 g/kg | | |
| Obacunone glycoside | Obacunone glycoside 0.02 g/kg + | 6.6 ± 1.3** | 13.65 ± 2.18** |
| combination | Dapagliflozin 0.02 g/kg | | |

Note
**after independent t-test, compared with the model group, the difference was extremely significant (P < 0.01)
*after independent t-test, compared with the model group, the difference was extremely significant (P < 0.05)

Discussion of Experimental Results

From the above results, it could be seen that, compared with the model group, either in single administration or in combination administration with dapagliflozin, obacunone and derivatives thereof could significantly reduce the blood glucose levels in the db/db diabetic mice. When obacunone and derivatives thereof were administrated in combination with dapagliflozin, significantly improved effect was observed relative to the single administration thereof, showing a synergistic effect. In addition, when obacunone and derivatives thereof were administrated in combination with dapagliflozin, as compared with their single administration, the doses of both could be effectively reduced while comparable glucose-lowering effects could still be achieved, which improved the safety of therapeutic regimen and reduced side effects.

Meanwhile, the limonoid compound represented by obacunone and its derivatives could significantly improve the sensitivity to leptin; and especially when administrated in combination with dapagliflozin, it could significantly improve the utilization efficiency of leptin in the body, improve the glucose metabolism of the body, and improve the functions relevant to the glucose metabolism in diabetes mice.

### EXAMPLE 3

Effects of limonoid compound, empagliflozin or combination thereof on blood glucose in mouse pancreatic islet β-cell injury model.

In this example, a mouse pancreatic islet β-cell injury model was established by modeling ICR mice with streptozotocin (STZ) (Li Nan et al., Protective effect of pine pollen on kidney damage in diabetic nephropathy mice, Science and Technology Review, 2014, 32 (4/5): 95-99), and used to complete the evaluation of hypoglycemic effect in animals (this model could simulate pancreatic islet β-cell damage state of type I and type II diabetics). The limonoid compound was selected from the group consisting of ichangin, ichangensin, and ichangin glycoside, and empagliflozin single administration group, ichangin single administration group, ichangensi single administration group, ichangin glycoside singe administration group, and respective combination thereof with empagliflozin administration groups were set.

Conditions of experimental feeding: ICR mice (20±2 g), aged 6 weeks, purchased from Zhejiang Academy of Medical Sciences, and subjected to experimental feeding after 7 days of preliminary feeding. It should be noted that the conditions for raising the mice were as follows: the temperature was 23±1° C., the humidity was 55±10%, the lights were turned on between 7 am and 7 pm (the lights were turned off at other time), and the mice were allowed to freely take in water and feed. The experimental feed was mouse growth-stable feed (GB M2118), and the daily feeding and management of the animals were under the responsibility of the animal security department, which provided the animals with sufficient diet and fresh drinking water daily.

Experimental grouping: 15 male mice were randomly selected as the normal control group. After fasting for 12 hours, the remaining mice were intraperitoneally injected once with STZ at a dose of 150 mg/kg, and 72 hours later, the mice with blood glucose value of 15 to 25 mmol/L were undifferentiatedly grouped and used in the experiment, 15 animals in each group, and subjected to blood sampling and detection of indicators after two weeks of administration.

Gavage doses: the gavage dose was 0.1 g/kg per day for the ichangin group, the gavage dose was 0.1 g/kg per day for the ichangensin group, the gavage dose was 0.1 g/kg per day for the ichangin glycoside group, the gavage dose of empagliflozin was 0.1 g/kg per day for the empagliflozin group, ichangin at a dose of 0.05 g/kg and empagliflozin at a dose of 0.05 g/kg were simultaneously gavaged per day for the ichangin/empagliflozin combination group, ichangensin at a dose of 0.05 g/kg and empagliflozin at a dose of 0.05 g/kg were simultaneously gavaged per day for the ichangensin/empagliflozin combination group, ichangin glycoside at a dose of 0.05 g/kg and empagliflozin at a dose of 0.05 g/kg were simultaneously gavaged per day for the ichangin glycoside/empagliflozin combination group, the gavage volume was 10 mL/kg, and the normal group and the model group were administrated with 10 mL/kg of distilled water. Two weeks later, the blood glucose values were measured by tail trimming method (Johnson's blood glucose meter) 1 h after the last administration, and the average of each group was obtained. SPSS 16.0 software was used for statistical analysis. The data were expressed as mean and standard deviation. The data before and after were analyzed by t-test, and P<0.05 was considered statistically significant. The test results were shown in Table 3 below.

TABLE 3
Blood glucose values of STZ mice after two weeks of daily intragastric gavage.

| | Formulation and dosage of | Blood glucose |
|---|---|---|
| Group | administration | value (mmol/L) |
| Normal control group | None | 7.5 ± 0.6 |
| Model group | None | 29.5 ± 4.6 |
| Empagliflozin group | Empagliflozin 0.1 g/kg | 13.9 ± 2.0** |
| Ichangin group | Ichangin 0.1 g/kg | 14.8 ± 1.9** |
| Ichangensin group | Ichangensin 0. l g/kg | 14.5 ± 3.5** |
| Ichangin glycoside group | Ichangin glycoside 0.1 g/kg | 14.9 ± 2.1** |
| Ichangin combination group | Ichangin 0.05 g/kg + | 7.5 ± 1.2** |
| | Empagliflozin 0.05 g/kg | |
| Ichangensin combination group | Ichangensin 0.05 g/kg + | 7.7 ± 1.5** |
| | Empagliflozin 0.05 g/kg | |
| Ichangin glycoside combination | Ichangin glycoside 0.05 g/kg + | 7.9 ± 0.9** |
| group | Empagliflozin 0.05 g/kg | |

Note
**After independent t-test, compared with the model group, the difference was extremely significant (P < 0.01)
*After independent t-test, compared with the model group, the difference was extremely significant (P < 0.05)

Discussion of Experimental Results

From the above results, it could be seen that, compared with the model group, either in single administration or in combination administration with empagliflozin, the three limonoid compounds all could significantly lower the blood glucose levels in the mice of the STZ pancreatic islet cell injury model. When they were administrated in combination with empagliflozin, their effects were significantly increased as compared with their single administration, similar to the normal mice in blood glucose level, showing a synergistic effect. In addition, when the above three limonoid compounds were administrated in combination with empagliflozin, as compared with their single administration, the doses of both could be effectively reduced while comparable glucose-lowering effects could still be achieved, which improved the safety of therapeutic regimen and reduced side effects.

### EXAMPLE 4

Effects of limonoid compound, ipragliflozin or combination thereof on blood glucose and insulin in mouse type II diabetes model.

In the present embodiment, the limonoid compound was selected nomilin, deacetylnomilin, nomilin acid, deacetylnomilin acid glycoside, and ipragliflozin single administration group, nomilin single administration group, deacetylnomilin single administration group, nomilin acid single administered group, deacetylnomilin acid glycoside single administration group, and respective combination thereof with ipragliflozin administration groups were set.

Conditions for experimental feeding: as type II diabetes model mice, 6-week-old SPF-grade db/db mice were purchased from the Nanjing Model Biology Institute, and subjected to experimental feeding after 7 days of preliminary feeding. It should be noted that the conditions for raising the mice were as follows: the temperature was 23±1° C., the humidity was 55±10%, the lights were turned on between 7 am and 7 pm (the lights were turned off at other time), and the mice were allowed to freely take in water and feed. The experimental feed was mouse growth-stable feed (GB M2118), and the daily feeding and management of the animals were under the responsibility of the animal security department, which provided the animals with sufficient diet and fresh drinking water daily.

Experimental grouping: male db/db mice (20±2 g) were selected, 18 male mice in each group were tested, and drinking bottles were sterilized weekly. The experimental groups included normal control group (db/m, n=18), model group (db/db, n=18), nomilin group (db/db, n=18), deacetylnomilin group (db/db, n=18), nomilin acid group (db/db, n=18), deacetylnomilin acid glycoside group (db/db, n=18), ipragliflozin group (db/db, n=18), nomilin combination group (db/db, n=18), deacetylnomilin combination group (db/db, n=18), nomilin acid combination group (db/db, n=18), deacetylnomilin acid glycoside combination group (db/db, n=18).

Gavage doses: nomilin at a dose of 0.2 g/kg was gavaged per day for the nomilin group, deacetylnomilin at a dose of 0.2 g/kg was gavaged per day for the deacetylnomilin group, nomilin acid at a dose of 0.2 g/kg was gavaged per day for the nomilin acid group, deacetylnomilin acid glycoside at a dose of 0.2 g/kg was gavaged per day for the deacetylnomilin acid glycoside group, ipragliflozin at a dose of 0.2 g/kg was gavaged per day for the ipragliflozin group, nomilin at a dose of 0.1 g/kg and ipragliflozin at a dose of 0.1 g/kg were simultaneously gavaged per day for the nomilin combination group, nomilin acid at a dose of 0.1 g/kg and ipragliflozin at a dose of 0.1 g/kg were simultaneously gavaged per day for the nomilin acid combination group, deacetylnomilin at a dose of 0.1 g/kg and ipragliflozin at a dose of 0.1 g/kg were simultaneously gavaged per day for the deacetylnomilin combination group, deacetylnomilin acid glycoside at a dose of 0.1 g/kg and ipragliflozin at a dose of 0.1 g/kg were simultaneously gavaged per day for the deacetylnomilin acid glycoside combination group, the gavage volume was 10 mL/kg, and the normal group and the model group were administrated with 10 mL/kg of distilled water. Two weeks later, the blood glucose values were measured by tail trimming method (Johnson's blood glucose meter) 1 h after the last administration, and serum insulin levels were measured with blood collected from orbital cavity by enzyme-linked immunosorbent assay (Elisa), and the average of each group was obtained. SPSS 16.0 software was used for statistical analysis. The data were expressed as mean and standard deviation. The data before and after were analyzed by t-test, and P<0.05 was considered statistically significant. The test results were shown in Table 4 below.

TABLE 4
Blood glucose values and insulin of db/db mice after two weeks of daily gavage.

| | Formulation and dosage of | Blood glucose | Insulin |
|---|---|---|---|
| Group | administration | value (mmol/L) | (pg/ml) |
| Normal control group | None | 6.0 ± 0.7 | 1.05 ± 0.39 |
| Model group | None | 23.9 ± 4.2 | 10.56 ± 3.00 |
| Ipragliflozin group | Ipragliflozin 0.2 g/kg | 10.3 ± 2.3** | 7.61 ± 1.03** |
| Nomilin group | Nomilin acid 0.2 g/kg | 10.1 ± 3.9** | 8.82 ± 3.42** |
| Nomilin acid group | Nomilin acid 0.2 g/kg | 11.0 ± 4.1** | 8.08 ± 1.49** |
| Deacetylnomilin group | Deacetylnomilin 0.2 g/kg | 11.9 ± 2.9** | 8.59 ± 2.61** |
| Deacetylnomilin acid | Deacetylnomilin acid | 12.8 ± 5.5** | 8.40 ± 3.27** |
| glycoside group | glycoside 0.2 g/kg | | |
| Nomilin combination group | Nomilin 0.1 g/kg + | 6.0 ± 1.2** | 3.65 ± 0.78** |
| | Ipragliflozin 0.1 g/kg | | |
| Nomilin acid combination | Nomilin acid 0.1 g/kg + | 6.2 ± 1.3** | 4.97 ± 0.85** |
| group | Ipragliflozin 0.1 g/kg | | |
| Deacetylnomilin combination | Deacetylnomilin 0.1 g/kg + | 6.3 ± 1.0** | 5.10 ± 0.90** |
| group | Ipragliflozin 0.1 g/kg | | |
| Deacetylnomilin acid | Deacetylnomilin acid | 6.5 ± 1.2** | 4.63 ± 0.72** |
| glycoside combination group | glycoside 0.1 g/kg + | | |
| | Ipragliflozin 0.1 g/kg | | |

Note
**after independent t-test, compared with the model group, the difference was extremely significant (P < 0.01)
*after independent t-test, compared with the model group, the difference was extremely significant (P < 0.05)

Discussion of Experimental Results

From the above results, it could be seen that, compared with the model group, either in single administration or in combination administration with ipragliflozin, nomilin and derivatives thereof could significantly reduce the blood glucose levels in the db/db diabetic mice. When nomilin and derivatives thereof were administrated in combination with ipragliflozin, significantly improved effect was observed relative to the single administration thereof, showing a synergistic effect. In addition, when nomilin and derivatives thereof were administrated in combination with ipragliflozin, as compared with their single administration, the doses of both could be effectively reduced while comparable glucose-lowering effects could still be achieved, which improved the safety of therapeutic regimen and reduced side effects.

Meanwhile, the limonoid compound represented by nomilin and derivatives thereof could significantly improve the sensitivity to insulin; and especially when administrated in combination with ipragliflozin, it could significantly improve the utilization efficiency of insulin in the body, improve the glucose metabolism of the body, and improve the functions relevant to the glucose metabolism in diabetes mice.

### EXAMPLE 5

Effects of limonoid compound, tofogliflozin or combination thereof on blood glucose in mouse type II diabetes model with pancreatic islet damage and obesity.

In this example, a mouse model of type II diabetes with pancreatic islet damage and obesity was established by multiple modeling ICR mice with a small dose of streptozotocin (STZ), following with continuous high-fat diets (referring to literature: Zhang Jiyuan et al, Study on the effect of three plant extracts on improving glucose and lipid metabolism in type 2 diabetic mice, Food and Machinery, 2016, 32 (12): 142-147). The limonoid compound was selected from the group consisting of nomilin glycoside, deacetylnomilin glycoside, and nomilin acid glycoside, and tofogliflozin single administration group, nomilin glycoside single administration group, deacetylnomilin single administration group, nomilin acid glycoside single administration group, and respective combination thereof with tofogliflozin administration groups were set.

Conditions of experimental feeding: ICR mice (20±2 g), aged 6 weeks, purchased from Zhejiang Academy of Medical Sciences, and subjected to experimental feeding after 7 days of preliminary feeding. It should be noted that the conditions for raising the mice were as follows: the temperature was 23±1° C., the humidity was 55±10%, the lights were turned on between 7 am and 7 pm (the lights were turned off at other time), and the mice were allowed to freely take in water and feed. The experimental feed was mouse growth-stable feed (GB M2118), and the daily feeding and management of the animals were under the responsibility of the animal security department, which provided the animals with sufficient diet and fresh drinking water daily.

Experimental grouping: 15 male mice were randomly selected as the normal control group, and the remaining mice were subjected to a high-fat diet (high-fat diet formula: cholesterol 1%, egg yolk powder 10%, lard oil 10%, and basic feed 79%, for establishing an obesity mouse model) for consecutive 4 weeks and intraperitoneal injection of STZ at a dose of 35 mg/kg for three consecutive days. After one week, the mice were subject to 24 hours of fasting and water deprivation, their fasting blood glucose was measured, and the mice with a blood glucose level of 15 to 25 mmol/L were selected and undifferentiatedly grouped and used in the experiment, continuously subjected to the high-fat diet, 15 mice in each group, and subjected to blood sampling and detection of indicators after 2 weeks of administration.

Gavage doses: the gavage dose was 0.5 g/kg per day for the nomilin glycoside group, the gavage dose was 0.5 g/kg per day for the deacetylnomilin glycoside group, the gavage dose was 0.5 g/kg per day for the nomilin acid glycoside group, tofogliflozin at a dose of 0.5 g/kg was gavaged per day for the tofogliflozin group, nomilin glycoside at a dose of 0.25 g/kg and tofogliflozin at a dose of 0.25 g/kg were simultaneously gavaged per day for the nomilin glycoside/tofogliflozin combination group, deacetylnomilin glycoside at a dose of 0.25 g/kg and tofogliflozin at a dose of 0.25 g/kg were simultaneously gavaged per day for the deacetylnomilin glycoside/tofogliflozin combination group, nomilin acid glycoside at a dose of 0.25 g/kg and tofogliflozin at a dose of 0.25 g/kg were simultaneously gavaged per day for the nomilin acid glycoside/tofogliflozin combination group, the gavage volume was 10 mL/kg, and the normal group and the model group were administrated with 10 mL/kg of distilled water. Two weeks later, the blood glucose values were measured by tail trimming method (Johnson's blood glucose meter) 1 h after the last administration, and the average of each group was obtained. SPSS 16.0 software was used for statistical analysis. The data were expressed as mean and standard deviation. The data before and after were analyzed by t-test, and P<0.05 was considered statistically significant. The test results were shown in Table 5 below.

TABLE 5
Blood glucose values of STZ mice after two weeks of daily intragastric gavage.

| | Formulation and dosage of | Blood glucose |
|---|---|---|
| Group | administration | value (mmol/L) |
| Normal control group | None | 5.9 ± 0.54 |
| Model group | None | 29.6 ± 6.0 |
| Tofogliflozin group | Tofogliflozin 0.5 g/kg | 10.1 ± 2.7** |
| Nomilin glycoside group | Nomilin glycoside 0.5 g/kg | 12.8 ± 2.9** |
| Deacetylnomilin glycoside group | Deacetylnomilin glycoside 0.5 g/kg | 12.7 ± 2.5** |
| Nomilin acid glycoside group | Nomilin acid glycoside 0.5 g/kg | 13.5 ± 2.4** |
| Nomilin glycoside combination | Nomilin glycoside 0.25 g/kg + | 5.9 ± 1.0** |
| group | Tofogliflozin 0.25 g/kg | |
| Deacetylnomilin glycoside | Deacetylnomilin glycoside 0.25 g/kg + | 5.7 ± 0.6** |
| combination group | Tofogliflozin 0.25 g/kg | |
| Nomilin acid glycoside | Nomilin acid glycoside 0.25 g/kg + | 5.9 ± 0.9** |
| combination group | Tofogliflozin 0.25 g/kg | |

Note
**After independent t-test, compared with the model group, the difference was extremely significant (P < 0.01)
*After independent t-test, compared with the model group, the difference was extremely significant (P < 0.05)

Discussion of Experimental Results

From the above results, it could be seen that, compared with the model group, either in single administration or in combination administration with tofogliflozin, the three limonoid glycosides all could significantly lower the blood glucose levels in the mice of the STZ type II diabetes model. When they were administrated in combination with tofogliflozin, their effects were significantly increased as compared with their single administration, similar to the normal mice in blood glucose level, showing a synergistic effect. In addition, when the above three limonoid glycosides were administrated in combination with tofogliflozin, as compared with their single administration, the doses of both could be effectively reduced while comparable glucose-lowering effects could still be achieved, which improved the safety of therapeutic regimen and reduced side effects.

### EXAMPLE 6

Method for preparing a tablet containing combination product of nomilin and canagliflozin.

In this example, a method for preparing a tablet of a combination product (nomilin and canagliflozin) of the present invention was exemplarily provided. A single tablet contained the following ingredients: 50 mg of nomilin, 400 mg of canagliflozin hydrochloride, 20 mg of hydroxypropylmethylcellulose, 30 mg of sodium carboxymethylcellulose, and 20 mg of microcrystalline cellulose, 5.2 mg of magnesium stearate, 20.8 mg of Opadry, and there were a total of 1000 tablets.

The preparation method comprised the following steps:

a) dissolving 50 g of nomilin in 5 L of 50% ethanol;

b) passing the raw and auxiliary materials through 100 mesh sieves, leaving them on standby;

c) weighing 400 g of canagliflozin hydrochloride, 20 g of hydroxypropylmethylcellulose, 30 g of sodium carboxymethylcellulose, and 20 g of microcrystalline cellulose, placing in a fluidized bed, and setting an inlet air volume of 500±50 m³/h, an inlet air temperature of 90±5° C., and a product temperature of 70±5° C., to perform hot melt granulation;

d) spraying a nomilin solution into the fluidized bed, setting an atomizing pressure of 1.0±0.2 bar, and a spraying speed of 30±10 Hz, to perform one-step granulation;

e) passing the resultant granules through a 1.0 mm round-hole screen to perform dry granulation;

f) adding 5.2 g of magnesium stearate and mixing for 5 min;

g) tabletting by using a 17×8.5 mm oval puncher at a pressure of 15 KN;

h) dissolving 20.8 g of Opadry 85F32004 in distilled water at a ratio of 1:4, setting parameters of a coating pan as: bed temperature of 40±2° C., outlet air temperature of 48±2° C., atomizing pressure of 0.6 Mpa, pan speed of 7 rpm, spray volume of 120 g/min, to complete film coating.

1. A combination product, comprising a limonoid compound or a pharmaceutically acceptable derivative, ester, stereoisomer, salt or prodrug thereof, and a SGLT-2 inhibitor or a pharmaceutically acceptable derivative thereof.

2. The combination product according to claim 1, wherein the combination product is in the form of a pharmaceutical composition.

3. The combination product according to claim 1, wherein the limonoid compound or a pharmaceutically acceptable derivative, ester, stereoisomer, salt or prodrug thereof, and the SGLT-2 inhibitor or a pharmaceutically acceptable derivative thereof are each in the form of a separate preparation.

4. The combination product according to claim 3, wherein the limonoid compound or a pharmaceutically acceptable derivative, ester, stereoisomer, salt or prodrug thereof, and the SGLT-2 inhibitor or a pharmaceutically acceptable derivative thereof are administered simultaneously or sequentially.

5. The combination product according to claim 1, wherein the SGLT-2 inhibitor has an amount of 50 mg to 2000 mg.

6. The combination product according to claim 1, wherein the limonoid compound has an amount of 50 mg to 2000 mg.

7. The combination product according to claim 1, wherein the SGLT-2 inhibitor is selected from the group consisting of canagliflozin, dapagliflozin, empagliflozin, ipragliflozin, luseogliflozin and tofogliflozin, and the limonoid compound is one or more selected from the group consisting of limonin, isolimonic acid, 7a-limonol, obacunone, ichangin, ichangensin, nomilin, deacetylnomilin, nomilin acid, deacetylnomilin acid, citrusin, isoobacunoic acid and any glycoside derivatives thereof.

8. The combination product according to claim 7, wherein the combination product further comprises a pharmaceutically acceptable carrier, diluent, or excipient.

9. The combination product according to claim 8, wherein the combination product is in the form of tablet, capsule, granule, syrup, powder, lozenge, sachet, cachet, elixir, suspension, emulsion, solution, syrup, aerosol, ointment, cream and injection.

10. A use of the combination product according to claim 1 in manufacture of a medicament, wherein the medicament is used for the prevention and/or treatment of a disease associated with diabetes and metabolic syndrome.
